(19) United States
(12) Patent Application Publication
(10) Pub. No.: US 2005/0038315 A1
(43) Pub. Date: Feb. 17, 2005

(54) Motorized syringe particularly useful for intra-uterine insemination

(75) Inventors: Nachman Eckstein, Tel Aviv (IL); Ellahu Eliachar, Haifa (IL); Nir Lilach, Kfar Yehoshua (IL)

Correspondence Address: Sol Sheinbein;c/o Anthony Castorina, Suite 207, 2001 Jefferson Davis Highway, Arlington, VA, 22202, US

(73) Assignee: Slo-Flo Ltd.

(21) Appl. No.: 10/638,404

(22) Filed: Aug. 12, 2003

(51) Int. Cl. A61B 17/43

(52) U.S. Cl. 600/35; 604/151; 604/110

(57) ABSTRACT

A motorized syringe or pump for producing a controlled, slow-delivery of a fluid-like substance, includes: a housing having, at one end, an expansible-contractible chamber for receiving a quantity of the fluid-like substance and an outlet for discharging the substance upon the contraction of the chamber; and a drive at the opposite end of the housing including an electrical motor and a plunger for contracting the chamber to discharge the substance via the outlet. The housing is constituted of a first section housing the expansible-contractible chamber, and a second section housing the drive. The first housing section is attachable to and detachable from the second housing section to permit disposal of the first housing section after a one-time use. The drive includes a threaded sleeve fixed to the plunger, a threaded shaft engageable with the threaded sleeve for axially displacing the sleeve and the plunger upon rotation of the shaft, and a step-down transmission coupling the threaded shaft to the motor for rotating the shaft, and thereby for axially displacing the sleeve and plunger at a slow controlled rate upon the energization of the motor.

### FIELD AND BACKGROUND OF THE INVENTION

The present invention relates to a novel motorized syringe or pump particularly for producing a controlled, slow-delivery of a fluid-like substance over a prolonged period of time. While the invention is useful for producing a controlled slow-delivery of various types of medical preparations, it is especially useful for intra-uterine insemination, and is therefore described below with respect to this application. The invention, therefore, also relates to a method of intra-uterine insemination utilizing the novel motorized syringe.

Therapeutic insemination has been used as a treatment for infertile couples for many years, but its application has recently become more widespread because of the development of a technique to separate sperm from semen, called “sperm washing”. Intra-uterine insemination (IUI) is a form of therapeutic insemination using either the husband's (AIH) or donor (AID) sperm. It is performed by passing a sterile catheter through the cervical canal into the uterine cavity, and then injecting a sperm suspension directly into the uterine cavity. This procedure is performed as close as possible to the time of ovulation.

The conventional procedure at the present time is to rapidly inject a bolus of semen via a catheter into the uterine lumen. The procedure lasts 10-15 minutes, and is repeated 24 hours following the first procedure. The bolus technique, however, has a number of disadvantages, including: intra-uterine hyperspermia; possible adverse immunological effects; phagocytosis of the sperm cells; failure to incorporate the physiological continuous slow release of sperm from the cervical crypts; and the possibility of flushing the ovum back into the fallopian tube.

Various types of slow-delivery pumps have been proposed for delivering the sperm preparation at a slow rate over a prolonged period of time. These include: osmotic pumps, which effect the delivery by generating an osmotic pressure; liposomal pumps, which effect the drug delivery by the release of active material from a core by contact with a surrounding liquid; syringe pumps; and various other types of generated-gas driven pumps. Examples of these known slow-delivery pumps are described in our prior International Application PCT/IL01/00625, published as International Publication No. WO 02/07619 on Jan. 31, 2002, the contents of which are incorporated herein by reference, and also in U.S. Pat. Nos. 5,536,243, 5,562,654, 5,904,665, 5,242,406, 5,785,688, 5,800,420, 5,840,332, 5,848,991, 5,858,001 and 5,904,934, referred in that International Publication.

As a rule, however, the foregoing types of slow-delivery pumps are not capable of obtaining the high degree of control in the slow-rate delivery required for a successful intra-uterine insemination procedure.

Slow-delivery pumps have also been proposed for the controlled slow-delivery of other types of medical preparations, e.g., in cancer therapy, in the administration of insulin, in the delivery of antibiotics, etc. An example of an infusion pump that may be used for this purpose is described in U.S. Pat. No. 5,000,739. Such an infusion pump, however, is not suitable for an intra-uterine insemination procedure or other procedures requiring placement of the pump in the patient's body.

### OBJECTS AND BRIEF SUMMARY OF THE PRESENT INVENTION

An object of the present invention is to provide a novel motorized syringe or pump for producing a controlled, slow-delivery of a fluid-like substance such as to be particularly useful for intra-uterine insemination, but also capable of other uses. Another object of the invention is to provide a method of intra-uterine insemination utilizing the novel motorized syringe or pump.

According to one aspect of the present invention, there is provided a motorized syringe for producing a controlled, slow-delivery of a fluid-like substance, comprising: a housing having, at one end. an expansible-contractible chamber for receiving a quantity of the fluid-like substance, and an outlet for discharging the substance upon the contraction of the chamber; and a drive at the opposite end of the housing, the drive including an electrical motor and a plunger driven by the electrical motor for contracting the chamber to discharge the substance via the outlet. The housing comprises a first section housing the expansible-contractible chamber, and a second section housing the drive. The first housing section is attachable to and detachable from the second housing section to permit disposal of the first housing section, including the expansible-contractible chamber, after a one-time use.

The drive section may thus be re-used or disposed after one-time use, or both sections could be disposed after one-time use.

As will be described more particularly below, a motorized syringe constructed in accordance with the foregoing features is especially useful in intra-uterine insemination since it is capable of being located within the female patient's uterus, produces a controlled slow-delivery of the preparation (semen suspension) to the target area, and conveniently enables the procedure to be repeated by merely detaching the first housing section including the expansible-contractible chamber containing the substance to be delivered and attaching another housing section containing another expansible-contractible chamber and a fresh supply of the substance to be delivered.

According to further features in the described preferred embodiment, the two housing sections include threaded ends for conveniently attaching and detaching them with respect to each other. However, other attaching means could be used, such as a bayonet-pin and socket, spring-biased detents, etc. In addition, the first housing section includes a septum for conveniently filling the expansible-contractible chamber by injection, e.g., via an injection needle or the like.

According to still further features in the described preferred embodiment, the expansible-contractible chamber is defined by a bellows-type container receivable within the first housing section. Also, the outlet includes a flexible catheter tube of a length to deliver the discharged substance to a desired location.

In the described preferred embodiment, the housing is constructed of a bio-compatible material suitable for introduction into a cavity of a patient's body for delivering a medical preparation thereto at a slow rate for a prolonged period of time. The housing in that embodiment is sized and configured for introduction into the vagina of a human female, and includes a flexible catheter tube of a length for introduction into the uterus of the female.

According to another aspect of the present invention, there is provided a motorized syringe for producing a controlled, slow-delivery of a fluid-like substance, comprising: a housing having, at one end, an expansible-contractible chamber for receiving a quantity of the fluid-like substance, and an outlet for discharging the substance upon the contraction of the chamber; and a drive at the opposite end of the housing, the drive including an electrical motor and a plunger driven by the electrical motor for contracting the chamber to discharge the substance via the outlet; the drive including a threaded sleeve fixed to the plunger, a threaded shaft engageable with the threaded sleeve for axially displacing the sleeve and the plunger fixed thereto upon the rotation of the threaded shaft, and a step-down transmission coupling the threaded shaft to the motor for rotating the shaft, and thereby for axially displacing the sleeve and plunger fixed thereto at a slow rate upon the energization of the motor.

The threaded shaft thus serves as an advance screw which is rotated to axially advance the non-rotating plunger.

As will be described more particularly below, a motorized syringe or pump constructed in accordance with the foregoing features is capable of providing a controlled, slow-delivery of a fluid-like substance such as to make it especially useful for intra-uterine insemination, as well as for other applications. Accordingly, another aspect of the present invention is to provide a method of intra-uterine insemination utilizing the novel pump of the present invention.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of motorized syringe constructed in accordance with the present invention and including its separate control unit;

FIG. 2 illustrates the motorized syringe of FIG. 1, and its control unit, as used in an intra-uterine insemination procedure;

FIG. 3 is a two-dimensional cut-away section illustrating the motorized syringe of FIG. 1;

FIG. 4 is an enlarged longitudinal sectional view illustrating the motorized syringe of FIG. 1;

FIG. 5 is a three-dimensional view illustrating the drive unit and the disposable unit in the motorized syringe of FIG. 1, with the plunger of the drive unit in its retracted position;

FIG. 6 is a view corresponding to that of FIG. 5, but illustrating the plunger of the drive unit in its extended position; and

FIG. 7a, 7b and 7c illustrate the motorized syringe of FIG. 1 in the plunger start-position, mid-position and end position, respectively.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to-understand and practice the described invention. It is to be further understood that the embodiments described are for purposes of example only, and that the invention is capable of being embodied in other forms and applications than described herein.

### DESCRIPTION OF A PREFERRED EMBODIMENT

As indicated earlier, the drawings illustrate a motorized syringe or pump constructed in accordance with a preferred embodiment of the invention for producing a controlled, slow-delivery of a fluid-like substance over a prolonged period of time. The illustrated preferred embodiment is designed particularly for use in an intra-uterine insemination procedure, but it will be appreciated that the various features of the invention, as will be described more particularly below, can be used in other applications requiring a controlled slow-delivery of a fluid-like substance, such as for administrating insulin, antibiotics, cancer-therapy drugs, hormones, labor inducing drugs, etc.

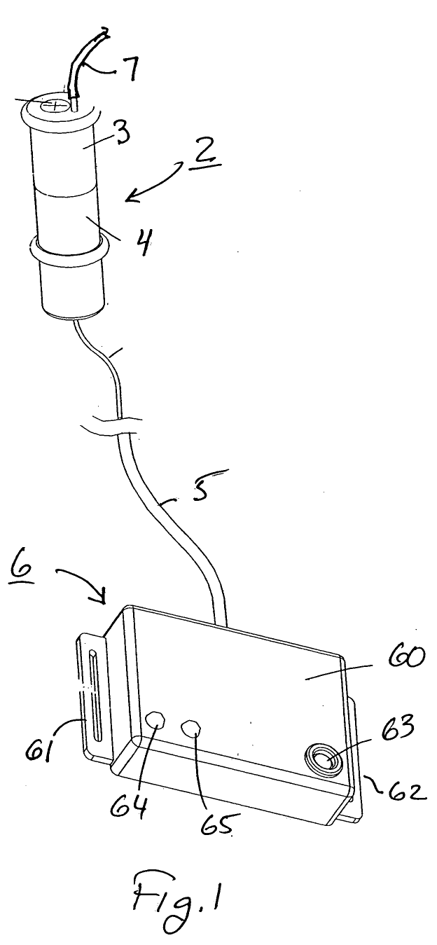

The motorized syringe illustrated in FIG. 1, therein generally designated 2, is constituted of two units: a preparation delivery unit 3, and a drive unit 4. The preparation delivery unit 3 is constructed so as to be a disposable one-time unit; whereas the drive unit 4 is constructed so as to be re-usable, but also disposable if so desired. As will be described more particularly below, the preparation delivery unit 3 includes an expansible-contractible chamber for receiving the preparation to be delivered; and the drive unit 4 includes a plunger or piston for contracting the chamber within unit 3 in order to effect a controlled slow-delivery of the preparation therefrom.

The drive unit 4 is coupled by a conductor 5 to a remotely-located control unit 6. A flexible catheter tube 7 is connected to the motorized syringe 2 and is of a length to deliver the preparation therefrom to a selected target area, e.g., the female patient's uterus.

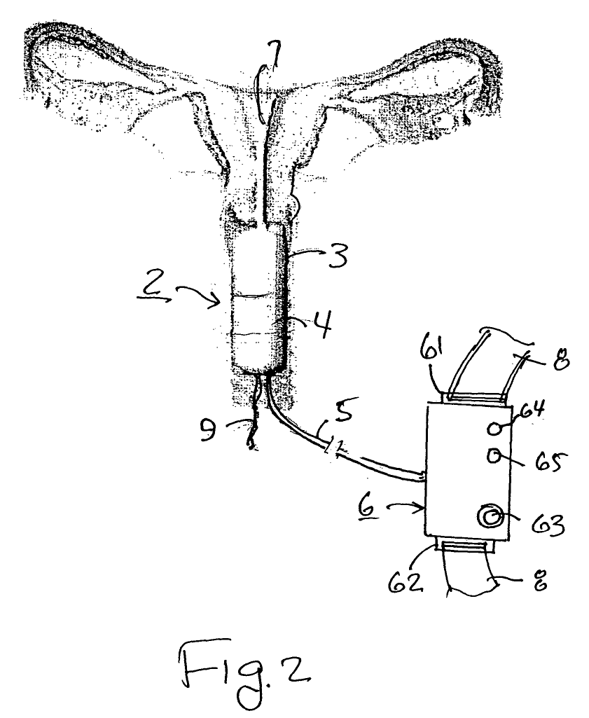

FIG. 2 illustrates the motorized syringe 2 of FIG. 1 and its separate control unit 6 as used in an intra-uterine insemination procedure. Thus, as shown in FIG. 2, motorized syringe 2, including its preparation delivery unit 3 containing a semen preparation to be delivered and its drive unit 4, is introduced into the vagina of the female patient; and the flexible catheter tube 7, connected to the output end of the preparation delivery unit 3, is introduced into the uterus of the female patient. These two units, therefore, would be made of a sterilizable bio-compatible material suitable for this purpose. The separate control unit 6, however, would be strapped to an external part of the patient being treated, e.g., to the patient's belt 8 shown in FIG. 2 or around the patient's thigh, and therefore need not be made of a bio-compatible material. Motorized syringe 2 may further include a pull-out cord, as shown at 9 in FIG. 2, to facilitate its removal when used in an intrauterine insemination procedure.

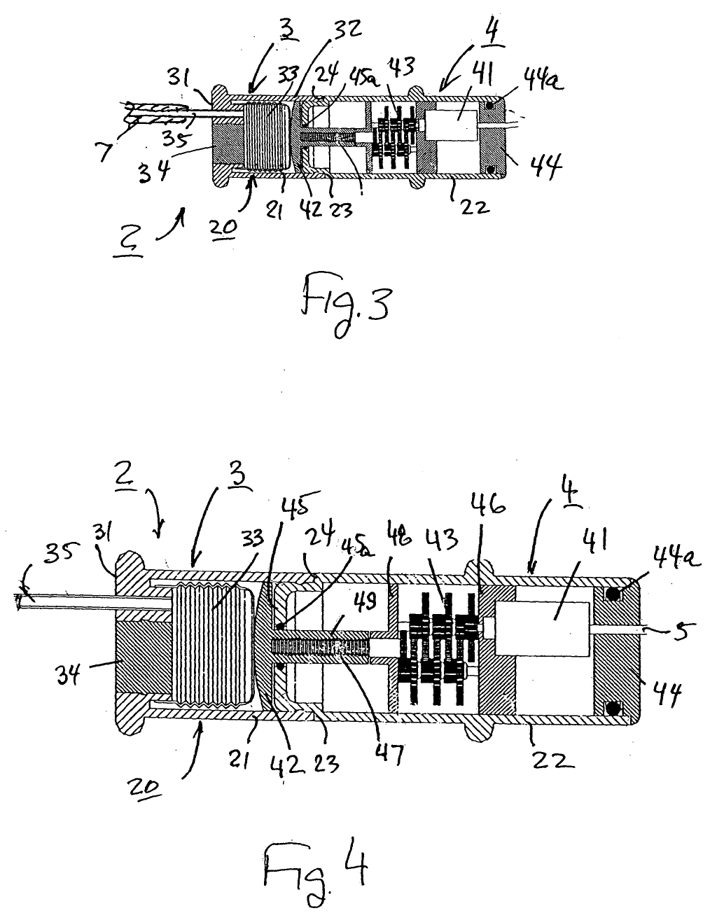

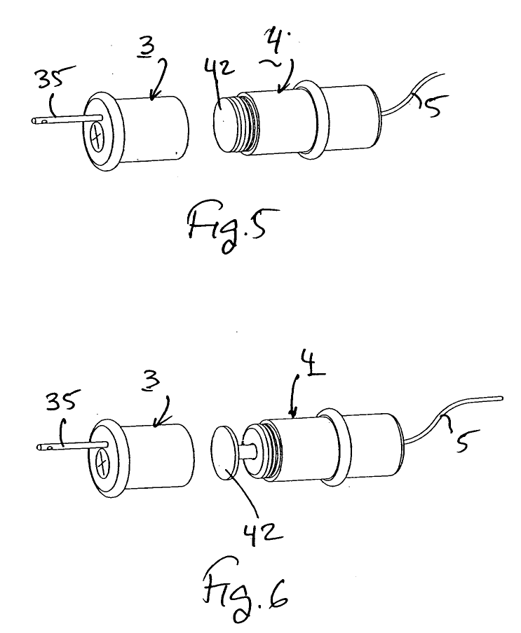

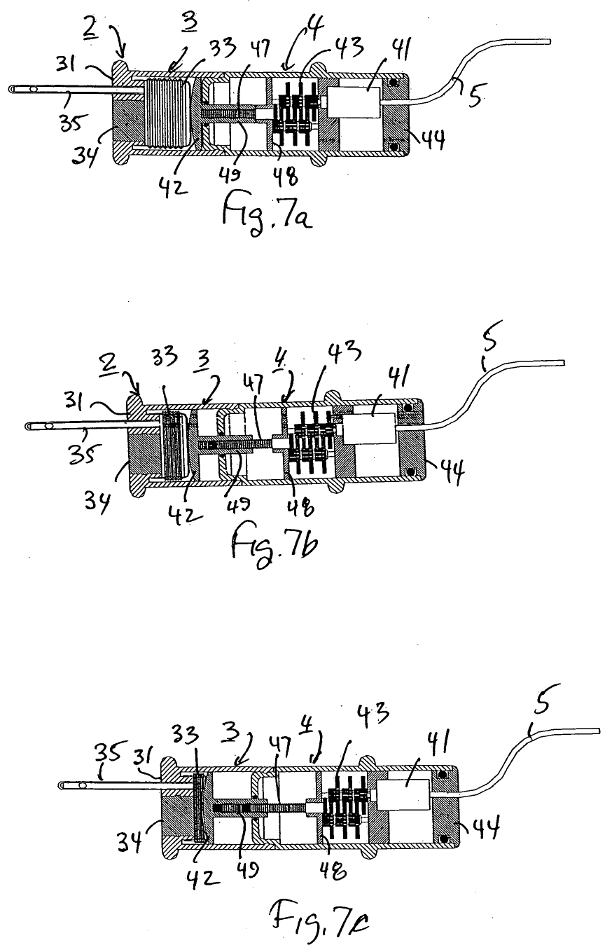

The internal structure of motorized syringe 2, and particularly of its preparation delivery unit 3 and its drive unit 4, is illustrated in FIGS. 3 and 4. FIGS. 5 and 6 illustrate the two units 3, 4 as being separable so as to permit the preparation delivery unit 3 to be disposed of after one-time use, while permitting re-use of the drive unit 4.

Thus, as shown in FIGS. 3-6, motorized syringe 2 includes a housing, generally designated 20, constituted of two sections 21, 22. One end of housing section 21 is formed with external threads 23, and the corresponding end of housing section 22 is formed with internal threads 24, to enable the two sections to be attachable and detachable with respect to each other in a convenient manner. Housing section 21 houses the components of the preparation delivery unit 3 to enable that unit to be disposed of after one-time use, whereas housing section 22 houses the components of the drive unit 4 such as to enable that unit to be re-used if so desired.

As shown in FIGS. 3 and 4, the disposable preparation delivery unit 3 includes an end wall 31 integrally formed at the end of its housing section 21, and a compartment 32 inwardly of end wall 31. Compartment 32 receives an expansible-contractible container 33, preferably of the bellows type, which defines an expansible-contractible chamber for receiving a quantity of the fluid-like substance to be delivered by the motorized syringe. In the preferred embodiment described herein wherein the motorized syringe is used for an intra-uterine insemination procedure, the fluid-like substance within the bellows container 33 would be a sperm suspension.

End wall 31 of the preparation delivery unit 3 further includes a septum 34 for conveniently introducing the fluid-like substance (sperm suspension) into bellows container 33 by injection. End wall 31 is further formed with a bore 35 for receiving the flexible catheter tube 7 (FIG. 1) which delivers the fluid-like preparation to the desired target area, in this case the patient's uterus.

It will thus be seen that when the preparation to be delivered by the motorized syringe is injected via septum 34 into the bellows container 33, the latter container is expanded. It will also be seen that the contraction of bellows container 33 will pump the preparation via the flexible catheter tube 7 to the target area (uterus) to receive the preparation.

Bellows container 33 is contracted in a precisely controllable manner by the drive unit 4. For this purpose, drive unit 4 includes, within housing section 22:a rotary electrical motor 41; a plunger or piston 42 driven in the axial direction by the electrical motor 41; and a step-down transmission, including step-down gearing 43, for converting the rotations of the output shaft of motor 41 to a very slow, controlled axial displacement of plunger 42. Plunger 42 engages the respective end wall of bellows container 33 to produce a corresponding controllable, slow contraction of the container, and thereby a controlled slow-delivery to the target area of the preparation within the container via catheter tube 7.

As shown particularly in FIG. 4, the drive unit 4 includes, end walls 44 and 45 at the opposite ends of housing section 22, and an; intermediate wall 46 cooperating with end wall 44 for supporting the rotary electrical motor 41. The two end walls are sealed by seals 44a, 45a, respectively.

The output shaft of electrical motor 41 passes through wall 46 and is coupled to one end of the step-down transmission 43. The opposite end of step-down transmission 43 is coupled to an externally-threaded shaft 47. Shaft 47 passes through another intermediate wall 48 and is received within an internally-threaded sleeve 49 passing through end wall 45 and fixed to plunger 42.

Wall 48 is fixed to wall section 22 defining the housing for the drive unit 4 and supports the externally-threaded shaft 47 at the output end of the step-down transmission 43. Rotation of shaft 47 will thus produce an axial movement of the internally-threaded sleeve 49, as well as of plunger 42. The movements of plunger 42 are thus non-rotary axial movements guided by threaded shaft 47 and seal 45a.

Electrical motor 41 within the drive unit 4 is connected by electrical conductor 5 to the separate control unit 6 (FIG. 1) which, as indicated earlier, can be at any desired remote location with respect to the drive unit 4, e.g., strapped to a belt on the patient's body, to the thigh of the patient's body, etc.

Control unit 6 (FIG. 1) thus includes a separate housing 60 integrally formed with a pair of strap connectors 61, 62 at its opposite sides for fixing the control unit to any suitable external part of the patient's body. Control unit 6 further includes an On/Off push-button switch 63 for energizing the control unit, and an indicator light 64 to indicate the “On” condition of the control unit. The control unit preferably includes its own power supply, such as batteries, and therefore preferably also includes another indicator 65 to indicate a “low-voltage” condition of the batteries.

Control unit 6 may include further features that may be desirable according to the particular application. For example, it could include a presettable device for presetting the rate of energization of the electrical motor, and thereby the rate of delivery of the preparation by the motorized syringe 2, as well as the period of its operation. The control unit may be factory programmed to operate at any desired rate. For example, the program can include various continuous rates, accelerating or decelerating rates, various pauses, etc, as desired for the particular application.

Preferably, electrical motor 41 within the drive unit 4 is a DC motor, but a digital step-motor may be utilized for higher resolution if required, to produce small increments of rotary movement of its output shaft. Such movements are stepped-down by the step-down transmission 43 and converted to axial movements by the externally-threaded shaft 47 and the internally-threaded sleeve 49.

The manner of using the described motorized syringe will be apparent from the above description. For purposes of example, the motorized syringe is described below for use in an intra-uterine insemination procedure:

The preparation and delivery unit 3, which as noted above is a separate. disposable unit for one-time use, is loaded with, e.g., 1 ml of a sperm suspension by injection through septum 34 in the end wall of unit 3. Unit 3 may thus be loaded with the sperm suspension while attached to the drive unit 4, or it may then be attached to the drive unit 4 via the threads 23, 24 at the respective ends of the two units. A catheter tube 7 is then attached to the preparation and delivery unit 3 by inserting its end into bore 35 and fixing it by glue. Unit 3, together with the drive unit 4 attached thereto, is introduced into the patient's vagina with the catheter 7 protruding into the patient's uterus. Any suitable inserter device may be used for this purpose, such as the inserter device described in the above-cited International Publication No. WO 02/07619.

Before the two units 3, 4 of motorized syringe 2 are introduced into the patient's vagina, the patient's cervix should be exposed by a bivalve speculum and then cleaned with a sponge. After exposing and creating an opening of the cervix and introducing the two units 3, 4 of motorized syringe 3 into the patient's vagina, the speculum is withdrawn allowing the uterus to collapse on the catheter. The inserter is then detached. The release of the inserter pushes the inserted device (units 3, 4 of pump 2) slightly forwardly, thereby better positioning it in the vagina and cervix.

The motorized syringe can then be activated by the separate remotely-located control unit 6. The patient may then be allowed to leave the clinic and carry on with her normal activities, but the patient should be instructed not to engage in strenuous physical activity or to bathe while the motorized syringe is operating.

Control unit 6 can be preset to energize the electrical motor 41 for a period of four hours, to gradually displace plunger 42, and thereby to effect a slow-delivery of the semen suspension via catheter 7 to the patient's uterus. After the factory-programmed work period (e.g., four hours) the motorized syringe automatically stops operating, but it is preferable to allow an additional hour before removing the motorized syringe from the patient. This can be conveniently done by using the electrical conductor 5, or a pull-out cord such as shown at 9 in FIG. 2, for this purpose.

Before leaving the clinic, the patient should be administered Doxycillin P.O. to prevent infection.

Preferably, the foregoing procedure is repeated after 24 hours. It may be further repeated, if necessary, until a pregnancy test indicates that the intra-uterine insemination procedure was successful.

Variations and Other Applications

While the invention has been described with respect to one preferred embodiment, it will be appreciated that this is set forth merely for purposes of example, and that the invention could be implemented with variations in the motorized syringe construction and in other applications. For example, the control unit 6 need not be made as a separate unit, but its components could be included in the drive unit 4, thereby obviating the need for a separate control unit. In addition, the drive unit 4 could also be disposable after one-time use. Further, other means could be used for attaching the preparation delivery unit 3 to the drive unit 4, such as by the use of a cick-on design (e.g., spring-biased pins carried by one unit received within recesses in the other unit), by a bayonnet-pin and socket arrangements, or by other partial-turn connector constructions.

Also, the preparation delivery unit 3 could include other types of expansible-contractible chambers for the preparation to be delivered, e.g., bladders or piston-type constructions. Also, the delivery rate and/or delivery time can be controlled as desired for the particular applications.

In addition, other step-down transmission arrangements could be used. For example, if the motor is a digital step motor stepped by digital pulses, a divider circuit could be used for producing an increment of movement for each tenth, hundredth, or even thousandth pulse.

Further, while the invention is particularly useful for intra-uterine insemination procedures, it will be appreciated that it could be used in many other applications requiring the controlled, slow-delivery of a fluid-like substance, such as for administering insulin, antibiotics, cancer-therapy drugs, hormones, labor inducing drugs and the like. Also, units 3 and 4 may be supplied as a single combined unit, while the control could be included in the combined unit or in the separate control unit 6.

Many other variations, modifications and applications of the invention will be apparent.

1. A motorized syringe for producing a controlled, slow-delivery of a fluid-like substance, comprising:
a housing having, at one end, an expansible-contractible chamber for receiving a quantity of said fluid-like substance, and an outlet for discharging said substance upon the contraction of said chamber;
and a drive at the opposite end of said housing, said drive including an electrical motor and a plunger driven by said electrical motor for contracting said chamber to discharge said substance via said outlet;
said housing comprising a'first section housing said expansible-contractible chamber and a second section housing said drive;
said first housing section being attachable to and detachable from said second housing section to permit disposal of said first housing section, including said expansible-contractible chamber after a one-time use.

2. The motorized syringe according to claim 1, wherein said first and second housing sections include interconnecting elements at one of their ends for attaching and detaching said housing sections with respect to each other in a quick manner.

3. The motorized syringe according to claim 2, wherein said interconnecting elements are external threads formed in one of said housing sections receivable in internal threads formed in the other of said housing sections.

4. The motorized syringe according to claim 1, wherein said first housing section includes a septum for filling said expansible-contractible chamber by injection.

5. The motorized syringe according to claim 1, wherein said expansible-contractible chamber is defined by a bellows-type container receivable within said first housing section.

6. The motorized syringe according to claim 1, wherein said outlet includes a flexible catheter tube of a length to deliver the discharged substance to a desired location.

7. The motorized-syringe according to claim 1, wherein said drive includes a threaded sleeve fixed to said plunger, a threaded shaft engageable with said threaded sleeve for axially displacing the sleeve and the plunger fixed thereto upon the rotation of said shaft, and a step-down transmission coupling said threaded shaft to said motor for rotating said shaft, and thereby for axially displacing said sleeve and plunger fixed thereto at a slow rate upon the energization of said motor.

8. The motorized syringe according to claim 1, wherein said housing is of a bio-compatible material suitable for introduction into a cavity of a patient's body for delivering a medical preparation thereto at a slow rate for a prolonged period of time.

9. The motorized syringe according to claim 8, wherein said motorized syringe further comprises a motor control circuit included in a separate unit having mounting means for mounting the separate unit externally of the patient's body.

10. The motorized syringe according to claim 8, wherein said housing is sized and configured for introduction into the vagina of a human female and includes a flexible catheter tube of a length for introduction into the uterus of the female.

11. A motorized syringe for producing a controlled, slow-delivery of a fluid-like substance, comprising:
a housing having, at one end, an expansible-contractible chamber for receiving a quantity of said fluid-like substance, and an outlet for discharging said substance upon the contraction of said chamber;
and a drive at the appropriate end of said housing, said drive including an electrical motor and a plunger driven by said electrical motor for contracting said chamber to discharge said substance via said outlet;
said drive including a threaded sleeve fixed to said plunger, a threaded shaft engageable with said threaded sleeve for axially displacing the sleeve and the plunger fixed thereto upon the rotation of the threaded shaft, and a step-down transmission coupling said threaded shaft to said motor for rotating said shaft, and thereby for axially displacing said sleeve and plunger fixed thereto at a slow rate upon the energization of said motor.

12. The motorized syringe according to claim 11, wherein said housing comprises:
a first section housing said expansible-contractible chamber, and a second section housing said drive;
said first housing section being attachable to and detachable from said second housing section to permit disposal of said first housing section, including said expansible-contractible chamber, after a one-time use.

13. The motorized syringe according to claim 12, wherein said first and second housing sections include interconnecting elements at one of their ends for attaching and detaching said housing sections with respect to each other in a quick manner.

14. The motorized syringe according to claim 12, wherein said first housing section includes a septum for filling said expansible-contractible chamber by injection.

15. The motorized syringe according to claim 12, wherein said expansible-contractible chamber is defined by a bellows-type container receivable within said first housing section.

16. The motorized syringe according to claim 11, wherein said outlet includes a flexible catheter tube of a length to deliver the discharged substance to a desired location.

17. The motorized syringe according to claim 11, wherein said housing is of a bio-compatible material for introduction into a cavity of a patient's body for delivering a medical preparation thereto at a slow rate for a prolonged period of time, and said motorized syringe further comprises a motor control circuit included in a separate unit having mounting means for mounting the separate unit externally of the patient's body.

18. The motorized syringe according to claim 17, wherein said housing is sized and configured for introduction into the vagina of a human female and includes a flexible catheter tube of a length for introduction into the uterus of the female.

19. A method of intra-uterine insemination, comprising:
introducing into the vagina of a female a motorized syringe according to claim 1, containing a semen preparation in its expansible-contractible chamber;
introducing into the uterus of the female a flexible catheter tube connected to the outlet of said motorized syringe;
and energizing said motorized syringe to deliver said semen preparation to said uterus at a slow flow rate for a prolonged period of time.

20. A method of intra-uterine insemination, comprising:
introducing into the vagina of a female a motorized syringe according to claim 11, containing a semen preparation in its expansible-contractible chamber;
introducing into the uterus of the female a flexible catheter tube connected to the outlet of said motorized syringe;
and energizing said motorized syringe to deliver said semen preparation to said uterus at a slow flow rate for a prolonged period of time.
